(19) United States
(12) Patent Application Publication
(10) Pub. No.: US 2012/0009257 A1
(43) Pub. Date: Jan. 12, 2012

(54) Galenical Formulations of a Fixed Dose Combination of Valsartan and Aliskiren

(76) Inventors: Indrajit Ghosh, East Hanover, NJ (US); Shoufeng Li, East Hanover, NJ (US); Wei-Qin Tong, Basking Ridge, NJ (US); Sudha Vippagunta, East Hanover, NJ (US); Hong Wen, East Hanover, NJ (US)

(21) Appl. No.: 13/256,454

(22) PCT Filed: Mar. 18, 2010
(86) PCT No.: PCT/US2010/027757

§ 371 Date: Sep. 14, 2011

Related U.S. Application Data
(60) Provisional application No. 61/161,891, filed on Mar. 20, 2009.

(51) Int. Cl. A61K 9/20; A61P 13/12; A61P 9/12; A61K 31/41

(52) U.S. Cl. 424/464; 514/381

(57) ABSTRACT

The invention provides a pharmaceutical oral fixed dose combination of aliskiren and valsartan. Aliskiren is shown to slow the dissolution rate of valsartan and the resultant undesirable gelling of valsartan in the presence of aliskiren is overcome by the use of disintegrants.

The present invention relates to pharmaceutical oral fixed dose combinations comprising an orally active renin inhibitor, Aliskiren, or a pharmaceutically acceptable salt thereof, and an angiotensin II antagonist, Valsartan, or a pharmaceutically acceptable salt thereof, as the active ingredients in a suitable carrier. In particular, the present invention provides galenical formulations comprising the hemi-fumarate salt of Aliskiren in combination with Valsartan. The present invention also relates to the processes for their preparation and to their use as medicaments.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure.

The renin inhibitor, Aliskiren, in particular, a hemi-fumarate thereof, is known to be effective in the treatment of reducing blood pressure irrespective of age, sex or race and is also well tolerated. Aliskiren in form of the free base is represented by the following formula

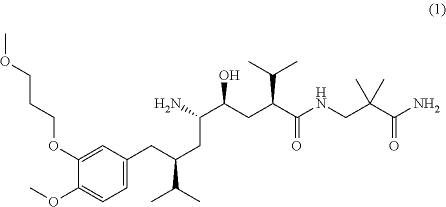

and chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. As described above, most preferred is the hemi-fumarate salt thereof which is specifically disclosed in EP 678503 A as Example 83.

Valsartan is a known Angiotensin receptor blocker (ARB, angiotensin II antagonist) and the combination with Aliskiren is described, e.g. in WO02/40007.

Angiotensin II is a hormone that causes blood vessels to constrict. This, in turn, can result in high blood pressure and strain on the heart. It is known that angiotensin II interacts with specific receptors on the surface of target cells. Two receptor subtypes for angiotensin II, namely AT1 and AT2, have been identified thus far. In recent times, great efforts have been made to identify substances that bind to the AT1 receptor. Angiotensin receptor blockers (ARBs, angiotensin II antagonists) are now known to prevent angiotensin II from binding to its receptors in the walls of blood vessels, thereby resulting in lower blood pressure. Because of the inhibition of the AT1 receptor, such antagonists can be used, therefore, as anti-hypertensives or for the treatment of congestive heart failure, among other indications.

Administration of such pharmaceutical agents via the oral route is preferred to parenteral administration because it allows self-administration by patients whereas parenteral formulations have to be administered in most cases by a physician or paramedical personnel.

However, Aliskiren is a drug substance difficult to formulate due to its physicochemical properties and it is not trivial to make oral formulations in the form of tablets in a reliable and robust way, in particular as regards physical properties of the tablet such as flowability, compression behavior or dissolution rate. For example, Aliskiren has a needle shaped crystallization habit, which has a negative influence on the bulk properties of the drug substance, e.g., flow properties and bulk density. The compression behavior of the drug substance is poor, leading to weak interparticulate bonds and polymorphism changes under pressure. Aliskiren has a strong elastic component that also leads to weakening of interparticulate bonds. The drug substance quality is very variable with effect on the proccessability of a tablet, e.g., particle size distribution, bulk density, flowability, wetting behavior, surface area and sticking tendency. Moreover, Aliskiren is highly hygroscopic. After contact with water and removal of the water, the drug substance polymorphism changes to an amorphous state, which shows inferior stability compared to the crystalline state. In addition, in the particular case of high dose of Aliskiren or a pharmaceutically acceptable salt thereof (up to 300 mg of the free base per tablet) makes a high drug loading necessary in order to achieve a reasonable tablet size.

The combination of these hurdles makes a standard tablet manufacturing process extremely difficult. A solid oral dosage form of Aliskiren is described in WO2005/089729.

On the other hand, Valsartan has pH dependent solubility whereby it ranges from very slightly soluble in an acidic environment to soluble in a neutral environment of the gastrointestinal tract. Further, development of a patient-convenient oral dosage form of Valsartan is challenging due to its low bulk density.

Moreover, in general the development of oral fixed dose combination formulations using certain active ingredients is challenging. In order to save time and costs in the development of a fixed dose combination, it is of advantage that the dissolution rate of any one of the active ingredients of the fixed dose combination and their respective dissolution profile in their free form (free dose combination) match. However, the development of fixed-dose combinations that have matching dissolution rate to the free dose combination may be challenging due to the multiplicity of hurdles arising from pharmacokinetic and pharmaceutical properties of the drugs sought to be combined.

The dissolution profile of valsartan may be slowed down in the presence of Aliskiren. Therefore, the impact of aliskiren on the dissolution rate of valsartan makes it desirable to develop formulations, in particular multilayer formulations, such as bilayer formulations, that overcome such a gelling issue of valsartan.

Surprisingly, it is found that disintegrants can play an important role in order to reach or achieve matching dissolution profile of valsartan in the presence of aliskiren to free valsartan, in particular in the form of compressed tablets, such as multi-layer tablets, in particular bilayer tablets. Thus, the present invention enables the manufacture of a pharmaceutical oral fixed dose combination comprising a therapeutically effective amount of Aliskiren, or a pharmaceutically acceptable salt thereof, and a therapeutically effective amount of Valsartan, or a pharmaceutically acceptable salt thereof, wherein a matching dissolution profile of valsartan in the presence of aliskiren to free valsartan is achieved or reached.

Throughout the present application, the various terms are as defined below:

The term “fixed dose combination” refers to a combination of defined doses of two drugs or active ingredients presented in a single dosage unit (e.g. a tablet or a capsule) and administered as such; further as used herein, “free dose combination” refers to a combination of two drugs or active ingredients administered simultaneously but as two distinct dosage units.

The term “disintegration” as used herein refers to a process where the pharmaceutical oral fixed dose combination, typically by means of a fluid, falls apart into separate particles and is dispersed. Disintegration is achieved when the solid oral dosage form is in a state in which any residue of the solid oral dosage form, except fragments of insoluble coating or capsule shell, if present, remaining on the screen of the test apparatus is a soft mass having no palpably firm core in accordance with USP<701>. The fluid for determining the disintegration property is water, such as tap water or deionized water. The disintegration time is measured by standard methods known to the person skilled in the art, see the harmonized procedure set forth in the pharmacopeias USP <701> and EP 2.9.1 and JP.

The term “dissolution” as used herein refers to a process by which a solid substance, here the active ingredients, is dispersed in molecular form in a medium. The dissolution rate of the active ingredients of the pharmaceutical oral fixed dose combination of the invention is defined by the amount of drug substance that goes in solution per unit time under standardized conditions of liquid/solid interface, temperature and solvent composition. The dissolution rate is measured by standard methods known to the person skilled in the art, see the harmonized procedure set forth in the pharmacopeias USP <711> and EP 2.9.3 and JP. For the purposes of this invention, the test is for measuring the dissolution of the individual active ingredients is performed following pharmacopoeia USP <711> at the pH as set forth herein for the different embodiments. In particular, at pH 4.5 and 1 the test is performed using a paddle stirring element at 75 rpm (rotations per minute). The dissolution medium is preferably a buffer, typically a phosphate buffer, especially one as described in the example “Dissolution Test”. The molarity of the buffer is preferably 0.1 M.

The term “physically separated” as defined herein refers to a pharmaceutical oral fixed dose combination containing both components a) and b) formulated such that they are not mixed with each other in the same carrier but are separated. This separation helps to minimize the interactions between the two components especially upon release of same. Typically the physical separation means that the two components a) and b) are present in different compartments, such as layers, or are present as different entities, such as particulates or granulates, of the formulation. It is not necessary that he two components a) and b) are further separated by additional layers or coating although this may be appropriate from case to case. This physical separation of the two components a) and b) in one dosage form can be achieved by various means known in the art. In one embodiment, this is achieved by formulating the respective components a) and b) into separate layers, e.g. a multi- or bilayer formulation. Specific examples of such formulation techniques are described hereinafter.

The terms “effective amount” or “therapeutically effective amount” refers to the amount of the active ingredient or agent which halts or reduces the progress of diabetic cardiomyopathy, or which otherwise completely or partly cures or acts palliatively on the condition.

The term “prophylactic ally effective amount” refers to the amount of the active ingredient or agent prevents the onset of diabetic cardiomyopathy.

The term “warm-blooded animal or patient” are used interchangeably herein and include, but are not limited to, humans, dogs, cats, horses, pigs, cows, monkeys, rabbits, mice and laboratory animals. In one embodiment, the mammals are humans.

The term “treatment” means the management and care of a patient for the purpose of preventing, combating or delaying progression of the disease, condition or disorder, preferably for the purpose of combating the disease, condition or disorder, and in particular it also prophylactic treatment.

The terms “prevention”/“preventing” are to be understood as meaning the prophylactic administration of a drug, such as a combined preparation or pharmaceutical composition, to healthy patients to prevent the outbreak of the disease, condition or disorder.

The terms “delay of progression”/“delaying progression” are to be understood as meaning the administration of a drug, such as a combined preparation or pharmaceutical composition, to patients being in a pre-stage of the disease, condition or disorder.

The term “aliskiren”, if not defined specifically, is to be understood both as the free base and as a salt thereof, especially the hemi-fumarate, nitrate, hydrogen sulfate and orotate salt thereof, in particular the hemi-fumarate salt thereof. Aliskiren, or a pharmaceutically acceptable salt thereof, can, e.g., be prepared in a manner known per se, especially as described in EP 678503 A, e.g., in Example 83.

The term “valsartan”, if not defined specifically, is to be understood both as the free base and as a salt thereof, especially a pharmaceutically acceptable salt thereof, as described below.

Valsartan, or a pharmaceutically acceptable salt thereof, can, e.g., be prepared in a manner known per se. Salts forms include acid addition salts. The compounds having at least one acid group (e.g., COOH or 5-tetrazolyl) can also form salts with bases. Suitable salts with bases are, e.g., metal salts, such as alkali metal or alkaline earth metal salts, e.g., sodium, potassium, calcium or magnesium salts, or salts with ammonia or an organic amine, such as morpholine, thiomorpholine, piperidine, pyrrolidine, a mono-, di- or tri-lower alkylamine, e.g., ethyl-, tert-butyl-, diethyl-, diisopropyl-, triethyl-, tributyl- or dimethylpropylamine, or a mono-, di- or trihydroxy lower alkylamine, e.g., mono-, di- or tri-ethanolamine. Corresponding internal salts may furthermore be formed. Salts which are unsuitable for pharmaceutical uses but which can be employed, e.g., for the isolation or purification of free compounds I or their pharmaceutically acceptable salts, are also included. In one embodiment, salts are, e.g., selected from the mono-sodium salt in amorphous form; di-sodium salt of Valsartan in amorphous or crystalline form, especially in hydrate form, thereof.

In one embodiment, salts are, e.g., selected from the calcium salt of Valsartan in crystalline form, especially in hydrate form, primarily the tetrahydrate thereof; magnesium salt of Valsartan in crystalline form, especially in hydrate form, primarily the hexahydrate thereof; calcium/magnesium mixed salt of Valsartan in crystalline form, especially in hydrate form; bis-diethylammonium salt of Valsartan in crystalline form, especially in hydrate form; bis-dipropylammonium salt of Valsartan in crystalline form, especially in hydrate form; bis-dibutylammonium salt of Valsartan in crystalline form, especially in hydrate form, primarily the hemihydrate thereof; mono-L-arginine salt of Valsartan in amorphous form; bis-L-arginine salt of Valsartan in amorphous form; mono-L-lysine salt of Valsartan in amorphous form; bis-L-lysine salt of Valsartan in amorphous form.

In one embodiment, Valsartan is used as the free acid.

The terms “drug,” “active substance,” “active ingredient” and “active agent” are to be understood as meaning a compound in free form or in the form of a pharmaceutically acceptable salt, in particular components a) or b) of the type specified herein. The active agents may be present in prodrug form. The invention includes prodrugs for the active pharmaceutical species of the invention, for example in which one or more functional groups are protected or derivatized but can be converted in vivo to the functional group, as in the case of esters of carboxylic acids convertible in vivo to the free acid, or in the case of protected amines, to the free amino group.

The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference. Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive alkylation
Nitro reduction.

Also to be mentioned as metabolic activations of prodrugs are nucleotide activation, phosphorylation activation and decarboxylation activation. For additional information, see “The Organic Chemistry of Drug Design and Drug Action,” R B Silverman (particularly Chapter 8, pages 497 to 546).

Where the plural form is used for compounds, salts, pharmaceutical compositions, diseases, disorders and the like, this is intended to mean one or more single compound(s), salt(s), pharmaceutical composition(s), disease(s), disorder(s) or the like, where the singular or the indefinite article (“a,” “an”) is used, this is intended to include the plural or the singular (“one”).

The term “polysaccharide” as used herein means a polymer made up of saccharide units.

The term “polysaccharide” is defined as being inclusive of homopolymers, copolymers of saccharide monomers and derivatives thereof, and it is inclusive of linear saccharide chains, non-linear saccharide chains and cross-linked saccharide chains.

The term “derivative thereof” refers to any chemically modified polysaccharide, wherein at least one of the monomeric saccharide units is modified by substitution of atoms or molecular groups or bonds. In one embodiment a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “copolymer” is defined as a polymer derived from more than one species of monomer, including copolymers that are obtained by copolymerization of two monomer species, those obtained from three monomers species (“terpolymers”), those obtained from four monomers species (“quaterpolymers”), etc. The term “copolymer” is further defined as being inclusive of random copolymers and alternating copolymers. The term “random copolymer” is defined as a copolymer comprising molecules in which the probability of finding a given monomeric unit at any given site in the chain is independent of the nature of the adjacent units. The term “alternating copolymer” is defined as a copolymer comprising molecules that include two species of monomeric units in alternating sequence.

The term “homopolysaccharide” as used herein means a polysaccharide made off a single type of saccharide unit. It is inclusive of non-linear and cross-linked polysaccharides. In one embodiment, a homopolysaccharide is a linear polysaccharide wherein the saccharide units are connected via alpha-glycosidic bonds or both alpha- and beta-glycosidic bonds. In another embodiment the term homopolysccharide is a linear polysaccharide wherein the saccharide unit is not glucose.

The term “heteropolysaccharide” as used herein means a polysaccharide wherein not all of the saccharide units are the same type. It is inclusive of linear, non-linear and cross-linked heteropolysaccharide.

The term “saccharide unit” as used herein means one saccharide molecule. A saccharide unit is a monomeric unit of a polysaccharide. The term “saccharide” is inclusive of carbohydrates, such as glucose, fructose or galactose, and derivatives thereof, such as mannuronic acid or guluronic acid.

The term “linear polysaccharide” as used herein means a polysaccharide whose saccharide units are arranged in chain-like fashion with no branches or bridges between the chains.

The term “cross-linked polysaccharide” as used herein means polysaccharide wherein there are bridges linking the polysaccharide chains.

The term “non-linear polysaccharide” or “branched polysaccharide” as used herein means a polysaccharide wherein there are saccharide units having at least one branching point, for example one to three branching points. This term is inclusive of any polysaccharide comprising at least one backbone and at least one terminal branch.

The term “branch” as used herein is inclusive of any saccharide unit or linear polysaccharide which is covalently attached at at least one end to the side group of a branching saccharide unit.

The term “L-HPC” or “LHPC” refers to low-substituted hydroxypropryl cellulose, wherein the term low-substituted means that there is of from 5% to 16% content of hydroxypropoxy groups (—OCH₂CHOHCH₃).

The terms “indigotin LAKE 12196” or “indigotin lake” or “indigotin farBlack” or “indigotin” refer to a coloring agent, pigment agent or dye commercially available, for example, from UNIVAR LTD and as described, for example, in www.kremer-pigmente.com and in http://www.foodadditivesworld.com/fdc-blue-no2-lake.html.

Release profile: The term “release” as used herein refers to a process by which the pharmaceutical oral fixed dose combination is brought into contact with a fluid and the fluid transports the drug(s) outside the dosage form into the fluid that surrounds the dosage form. The combination of delivery rate and delivery duration exhibited by a given dosage form in a patient can be described as its in vivo release profile. The release profiles of dosage forms may exhibit different rates and durations of release and may be continuous. Continuous release profiles include release profiles in which one or more active ingredients are released continuously, either at a constant or variable rate.

When two or more components that have different release profiles are combined in one dosage form, the resulting individual release profiles of the two components may be the same or different compared to a dosage form having only one of the components. Thus, the two components can affect each other's release profile leading to a different release profile for each individual component.

A two-component dosage form can exhibit release profiles of the two components that are identical or different to each other. The release profile of a two-component dosage form where each component has a different release profile may be described as “asynchronous”. Such a release profile encompasses both (1) different continuous releases where preferably component b) is released at a slower rate than component a), and (2) a profile where one of components a) and b), preferably component b), is released continuous and the other of components a) and b), preferably component a), is modified to be released continuous with a time delay. Also a combination of two release profiles for one drug is possible e.g. 50% of the drug in continuous and 50% of the same drug continuous with a time delay.

Immediate release: For the purposes of the present application, an immediate release formulation is a formulation showing a release of the active substance(s), which is not deliberately modified by a special formulation design or manufacturing method.

Modified release: For the purposes of the present application, a modified release formulation is a formulation showing a release of the active substance(s), which is deliberately modified by a special formulation design or manufacturing method. This modified release can be typically obtained by delaying the time of release of one or both of the components, preferably component a). Typically for the purposes of the present invention, a modified release refers to a release over 5 h, such as a release over 3 h or even shorter. Modified release as used herein is meant to encompass both a different continuous release over time of the two components or a delayed release where one of the components, preferably component a), is released only after a lag time. Such a modified release form may be produced by applying release-modifying coatings, e.g. a diffusion coating, to the drug substance(s) or to a core containing the drug substance(s), or by creating a release-modifying matrix embedding the drug substance(s).

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the dosage form and the release of the active ingredient from another component of the dosage form.

### SUMMARY OF THE INVENTION

In one embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
c) a disintegrant, and
d) one or more, for example one to three, disintegrant differing from the disintegrant c) and being a polysaccharide, provided that it is not cellulose, and

provided that if Indigotin lake is comprised in the composition it is not in an amount of 0.13, 0.2, 0.25 or 0.5 mg per unit dose.

Surprisingly, a matching dissolution profile of valsartan in the presence of aliskiren to free valsartan is achieved or reached by such a pharmaceutical oral fixed dose combination.

Preferred embodiments are as defined herein and in the subclaims.

In one embodiment, the polysaccharide component d) is selected from a non-linear, linear or crossed-linked heteropolysaccharide; a linear polysaccharide, wherein the saccharide units are connected via alpha-glycosidic bonds or both alpha- and beta-glycosidic bonds or are connected via beta-glycosidic bonds provided that the saccharide unit is not glucose; a non-linear homopolysaccharide and a crossed-linked homopolysaccharide. The polysaccharide component d) is preferably selected from a polycarboxyalkyl ether of cellulose or starch, such as a polycarboxymethyl ether, and a low-substituted poly(hydroxylalkyl)ether of cellulose, such as a poly(hydroxypropyl)ether; in particular croscarmellose sodium, sodium starch glycolate, alginate, starch, low-substituted hydroxypropyl cellulose, and pregelatinized starch. Most preferably component d) is low-substituted hydroxypropyl cellulose.

In one embodiment, the polysaccharide d) comprises uronic residues, such a polysaccharide is for example alginate.

In another embodiment, the polysaccharide d) is a cellulose derivative, such a polycarboxyalkyl ether of cellulose, for example polycarboxymethyl ether of cellulose, in particular croscarmellose sodium, or is a starch derivative, such a polycarboxyalkyl ether of starch, for example polycarboxymethyl ether of starch, in particular sodium starch glycolate.

In one embodiment, component c) is crospovidone.

The components a) to d) of the pharmaceutical composition of the present invention are preferably employed in the following weight ratios, based on the total weight of the pharmaceutical composition.

In a preferred embodiment of the present invention, component (a) is present in an amount ranging from 10 to 45%, such as 10 to 35%, by weight based on the total weight of the pharmaceutical oral fixed dose combination. These percentages refer to the hemifumarate salt of aliskiren, and if the free base or other salts are used, the percentages will be adapted accordingly.

In another preferred embodiment of the present invention component a) is present in an amount of from 12 to 45%, such as of from 12 to 40%, in one embodiment of from 12 to 30%, such as of from 12 to 25%, by weight based on the total weight of the pharmaceutical oral fixed dose combination. These percentages refer to the hemifumarate salt of aliskiren, and if the free base or other salts are used, the percentages will be adapted accordingly.

It is preferred that component (a) is present in an amount ranging of from 75 mg to 300 mg of the free base per unit pharmaceutical oral fixed dose combination, and if a salt is used, the amounts will be adapted accordingly.

In a preferred embodiment of the present invention, component (a) is present in an amount ranging from 75 to 300 mg, such as 75 to150 mg, of the free base per unit pharmaceutical oral fixed dose combination, in particular 75, 150 or 300 mg, such as 150 or 300 mg, and if a salt is used, the amounts will be adapted accordingly.

In a preferred embodiment of the present invention, component (b) is present in an amount ranging from 8 to 45%, such as 10 to 30%, in particular 12 to 27%, by weight based on the total weight of the pharmaceutical oral fixed dose combination. These percentages are based on the free acid of component b) and if a salt is used, the percentages will be adapted accordingly.

In a preferred embodiment of the present invention, component (b) is present in an amount of 20 to 40, such as 20 to 30%, by weight based on the total weight of the pharmaceutical oral fixed dose combination. These percentages are based on the free acid of component b) and if a salt is used, the percentages will be adapted accordingly.

It is preferred that component (b) is present in an amount ranging from 75 to 350 mg, such as 100 to 200 mg, more preferably 80 mg to 320 mg, such as 160 to 320 mg, per unit dosage form, in particular 80, 160 or 320 mg, such as 160 or 320 mg. and if a salt is used, the amounts will be adapted accordingly.

The weight ratio of component (a) to component (b) preferably ranges of from 1:0.001 to 1:5, more preferably of from 1:0.5 to 1:4 or 1:0.03 to 1:0.07. Most preferably, the weight ratio is of from 1:1.0 to1.1; 1:2.1 to 2.2; or 1:0.005 to 0.006 based on the free acids of (a) and (b). Most preferably, components (a) and (b), are used in amounts of 75/80 mg, 75/160 mg, 150/80 mg, 150/160 mg, 300/320 mg, 300/160 mg or 150/320 mg, most preferably 150/160 mg, 300/320 mg, 300/160 mg or 150/320 mg of (a)/(b), based on the free acids of (a) and (b). In one embodiment it is preferred to use a high drug load using 300 mg of (a) and/or 320 mg of (b), most preferably 300/320 mg of (a)/(b). When using a salt, such as the hemifumarate for component a), and/or a salt of component b) the ratios will be adapted accordingly.

In a preferred embodiment, the weight ratio of component b) to component d) is of from 15:1 to 2:1, preferably 8:1 to 2:1, more preferably 6:1 to 3:1. These ratios are based on the free acid of component b) and if a salt is used, the percentages will be adapted accordingly.

In a preferred embodiment, the weight ratio of component c) to component d) is of from 1:1 to 1:8, preferably of from 1:1 to 1:5, more preferably of from 1:1 to 1:3.

The pharmaceutical oral fixed dose combination according to the present invention needs to be selected appropriately to show the desired dissolution profile. Typically, the pharmaceutical oral fixed dose combination is a solid dosage form.

The oral fixed dose combination of the present invention preferably exhibits release profiles of both components a) and b), more preferably component a) that are regarded as modified release profiles. The oral fixed dose combination of the present invention preferably exhibits a release profile of component d) that is regarded as an immediate release profile. In a preferred embodiment of the present invention, the release profiles of the two active principles a) and b) of the oral fixed dose combination are asynchronous. In one embodiment, both components are released continuously with an asynchronous release profile, whereby one of the components, preferably component a), is modified to be released at a slower continuous rate. In another embodiment, one of the components, preferably component a), is released with a time delay so as result in a time lag of component a) compared to component b).

Preferably, the pharmaceutical oral fixed dose combination of the present invention is designed in such a way that components a) and b) are physically separated. Typical technologies and formulation principles for pharmaceutical oral fixed dose combinations capable to match the required dissolution profile according to the present invention include the formulation examples described below in more detail.

Pharmaceutically acceptable additives suitable for use in formulations according to the present invention, in particular multilayer tablets, in particular bilayer tablets, include, without limitation, diluents or fillers, disintegrants, glidants, lubricants, binders, colorants and combinations thereof. Preferred pharmaceutically acceptable additives include fillers and binders. The amount of each additive in a pharmaceutical oral fixed dose combination may vary within ranges conventional in the art.

Suitable fillers include, without limitation, microcrystalline cellulose (e.g., cellulose MK GR), mannitol, sucrose or other sugars, such as lactose, or sugar derivatives, calcium hydrogen phosphate, hydroxyethyl cellulose, and combinations thereof, preferably, microcrystalline cellulose, e.g., products available under the registered trade marks AVICEL, FILTRAK, HEWETEN or PHARMACEL. When present, one or more filler in the layer containing component a) may be employed. When present, the total amount of one or more filler is preferably an amount ranging of from 1% to 40%, preferably of from 10% to 30% by weight of the tablet (prior to any optional film coating). As regards the layer containing component b), when present, a filler may be employed in an amount ranging of from 1% to 40%, preferably of from 10% to 30% by weight of the tablet (prior to any optional film coating).

In multilayer tablets according to the present invention, preferably the filler or combination of fillers present in the layer comprising component b) is in an amount of not more than 40%, such as 1 to 40%, in particular 5 to 35%, specially 10 to 30% by weight of the tablet (prior to any optional film coating). In one embodiment, the layer comprising component b) contains microcrystalline cellulose, such as cellulose MK GR, in particular Avicel, preferably in an amount of not more than 40%, such as 1 to 40%, in particular 5 to 35%, specially 10 to 30% by weight of the tablet (prior to any optional film coating). Preferably, both layers contain a filler.

Suitable binders include, without limitation, polyvinylpyrrolidone (PVP), such as e.g., PVP K 30 or PVP9OF, polyethylene glycols (PEG), e.g., PEG 4000, hydroxypropylmethyl cellulose, hydroxypropyl cellulose, both preferably of medium to high viscosity, e.g., viscosity grades 3 or 6 cps and combinations thereof. A most preferred binder is PVP K 30 or PVP9OF. It was found that the presence of binder in the layer containing component a) plays an important role in obtaining the desired dissolution profile. A roller compacted layer containing component a) preferably contains the binder in the internal phase and a wet-granulated layer containing component a) preferably contains the binder in the internal and in the external phase. When present, a binder in the layer containing component a) may be employed in an amount ranging of from 0.1% to 20%, preferably of from 0.5% to 15%, such as 0.7% to 10%, by weight of the multilayer, preferably bilayer, tablet (prior to any optional film coating). When present, a binder in the layer containing component b) may be employed in an amount ranging of from 0.1% to 20%, preferably of from 0.2% to 10% by weight of the multilayer, preferably bilayer tablet, (prior to any optional film coating).

Suitable lubricants include, without limitation, magnesium stearate, aluminum or calcium silicate, stearic acid, cutina, PEG 4000-8000, talc and combinations thereof, preferably magnesium stearate. When present, a lubricant in the layer containing component a) may be employed in an amount ranging of from 0.1% to 5%, preferably of from 0.5% to 3%, by weight of the multilayer, preferably bilayer, tablet (prior to any optional film coating). When present, a lubricant in the layer containing component b) may be employed in an amount ranging of from 0.1% to 5%, preferably of from 0.5% to 3%, by weight of the multilayer, preferably bilayer tablet (prior to any optional film coating). Preferably, both layers contain a lubricant, in each case preferably both in the external and the internal phase.

Suitable disintegrants include, without limitation, carboxymethylcellulose calcium (CMC—Ca), carboxymethylcellulose sodium (CMC—Na), crosslinked PVP (e.g. CROSPOVIDONE, POLYPLASDONE or KOLLIDON XL), alginic acid, sodium alginate and guar gum, most preferably crosslinked PVP (CROSPOVIDONE), crosslinked CMC (Ac-Di-Sol), carboxymethylstarch-Na (PIRIMOJEL and EXPLOTAB). A most preferred disintegrant is crosslinked PVP, preferably PVPPXL. When present, a disintegrant in the layer containing component a) may be employed in an amount ranging of from 0.5% to 20%, preferably of from 1% to 3%, by weight of the multilayer, preferably bilayer, tablet (prior to any optional film coating). When present, a disintegrant in the layer containing component b) may be employed in an amount ranging of from 1% to 20%, preferably of from 2% to 12%, by weight of the multilayer, preferably bilayer tablet (prior to any optional film coating). Preferably the disintegrant is absent in the layer containing component a), especially in a roller compacted layer containing component a). A wet granulated layer containing component a) may contain the disintegrant. Preferably the layer containing component b) includes a disintegrant.

Suitable glidants include, without limitation, colloidal silicon dioxide (e.g., Aerosil 200), magnesium trisilicate, powdered cellulose, talc and combinations thereof. When present, a glidant in the layer containing component a) may be employed in an amount ranging of from 00.05% to 5%, preferably of from 0.1% to 1%, by weight of the multilayer, preferably bilayer, tablet (prior to any optional film coating). When present, a disintegrant in the layer containing component b) may be employed in an amount ranging of from 0.05% to 5%, preferably of from 0.1% to 1%, by weight of the multilayer, preferably bilayer tablet (prior to any optional film coating).

Multilayer Tablets

In one embodiment, the present invention is in particular related to a pharmaceutical oral fixed dose combination in the form of a multilayer tablet. A multilayer tablet has at least two layers (bilayer tablet) or can have three, four, five or more layers. Each of the layers contains not more than one of the components. Preferably, the tablet has two layers with one of the components in one of the two layers, but it is also possible that in addition to these two layers the tablet contains further layers containing only carrier and which may function e.g. as separation layer(s) or outer coating layer(s). Alternatively, if more than two layers are present, the components may be present in more than one layer as long as they are not present together in the same layer. For practical purposes, a bilayer tablet is preferred but all information detailed below is equally applicable to all multilayer tablets.

Multilayer tablets, in particular, bilayer tablets, according to the present invention are characterized in that one layer contains component a) and the other layer contains component b). In a preferred embodiment, the layer comprising component b) further comprises components c) and d).

A bilayer tablet comprising components a) and b) is described in PCT/US08/077416.

In a one embodiment, the pharmaceutical composition of the present invention in the form of a multilayer tablet, preferably bilayer tablet, comprises component d) in the layer containing component b). In this embodiment, component d) is in an amount of from 2 to 20%, preferably of from 4 to 10%, more preferably of from 4 to 6% by weight of the multilayer, preferably bilayer, tablet (prior to any optional film coating).

In another embodiment, the pharmaceutical composition of the present invention in the form of a multilayer tablet, preferably bilayer tablet, comprises a further disintegrant, component e), for example crospovidone, in the layer containing component a). In this embodiment, the weight ratio of component a) to component e) is of from 40:1 to 10:1, preferably of from 30:1 to 10:1, more preferably of from 30:1 to 15:1. The ratios refer to the free base of aliskiren and if a salt is used, the ratios will be adapted accordingly.

In a further embodiment, the weight ratio of component d), comprised in the layer comprising component b), to component e), comprised in the layer comprising component a), is of from 15:1 to 3:1, preferably of from 10:1 to 3:1, more preferably of from 7:1 to 3:1.

Multilayer tablets, in particular, bilayer tablets, can be manufactured by methods known in the art, in particular, the methods described for preparing the individual tablets containing either component a) or component b). Preferably, each of the layers can be prepared using wet granulation, melt extrusion or dry granulation. Examples for wet granulation are aqueous or organic wet granulation, in particular organic wet granulation. Preferred examples of dry granulation include roller compaction. Dry granulation methods are preferred since these circumvent the use of solvents and avoid additional drying steps. For the multilayer tablet, in particular, the bilayer tablet of the present invention, the individual layers can be prepared by the same or different processes for example one layer can be prepared by wet granulation and the second layer can be prepared by roller compaction. Most preferably, both layers are prepared by using roller compaction.

In one embodiment, the pharmaceutical oral fixed dose combinations of invention are multilayer, preferably bilayer, tablet pharmaceutical oral fixed dose combinations of low friability. Preferably the friability is not more than 0.8%. The friability is measured by standard methods known to the person skilled in the art, see the harmonized procedure set forth in the pharmacopeias USP <1216> and EP 2.9.7 and JP.

In another embodiment, the pharmaceutical oral fixed dose combinations according to the present are multilayer, preferably bilayer, tablet pharmaceutical oral fixed dose combinations of suitable hardness (e.g. an average hardness ranging of from 250 N to 300 N for bilayer forms). Such an average hardness is determined prior to the application of any film coating on the pharmaceutical oral fixed dose combinations. In that regard, a preferred embodiment of this invention is directed to pharmaceutical oral fixed dose combinations which are film-coated. Suitable film coatings are known and commercially available or can be made according to known methods. Typically the film coating material is a polymeric film coating material comprising materials such as polyethylene glycol, talc and colorant. Typically, a film coating material is applied in such an amount as to provide a film coating that ranges of from 1% to 6% by weight of the film-coated tablet.

A further embodiment of the present invention is a process for the manufacture of a multilayer, preferably a bilayer, tablet according to the present invention. For example, a bilayer tablet comprising one layer containing component a) and one layer containing component b) can be prepared by the following method, comprising the steps of (1) granulating component a) and pharmaceutically acceptable additives, optionally in the presence of a granulation liquid, to form an Aliskiren granulate; (2) granulating component b) and pharmaceutically acceptable additives to form a Valsartan granulate; (3) optionally drying resulting respective granulates; (4) sieving; (5) optionally mixing the respective granulates with outer phase excipients; and (6) compressing the Valsartan granulates and the Aliskiren granulates together to form a bilayer tablet. The details regarding the components a) and b) and pharmaceutically acceptable additives, i.e., source, amount, etc., are as set forth above. In one embodiment, the layer containing component a) is obtainable by roller compaction. In another embodiment, the layer containing component b) is obtainable by roller compaction.

In the first step of the method, component a) is granulated with pharmaceutically acceptable additives, optionally in the presence of a granulation liquid, optionally with component e), to form an Aliskiren granulate. The granulation liquid can be any liquid or liquid mixture well-known in the granulation art such as ethanol, a mixture of ethanol and water, a mixture of ethanol, water and isopropanol, said mixtures may contain a binder, such as those described herein. The process is then referred to as an organic wet granulation. A preferred mixture of ethanol and water ranges of from 50/50 to 99/1 (% w/w), most preferably it is 94/6 (% w/w). A preferred mixture of ethanol, water and isopropanol ranges of from 45/45/5 to 98/1/1 (% w/w/w), most preferably of from 88.5/5.5/6.0 to 91.5/4.5/4.0 (% w/w/w). In a preferred embodiment, the granulation is effected by an ethanolic solution of the binder and additional ethanol. Aliskiren granulation can be accomplished by any suitable means, for example wet granulation or melt extrusion granulation.

Aliskiren wet granulation is typically accomplished by using the following method (1) blending component a) and pharmaceutically acceptable additives in the presence of a granulation liquid to form a blended material; (2) drying the blended material, (3) sieving the blended material; and (4) screening the sieved material to isolate the adequate Aliskiren granulate fraction.

Alternatively, Aliskiren granulation is accomplished using another method (dry granulation) as follows: (1) blending component a) and pharmaceutically acceptable additives to form a blended material; (2) sieving the blended material; (3) blending the sieved material to form a final blend material; (4) compacting the final blend material to form a compacted material; (5) milling the compacted material to form a milled material; and (6) blending the milled material to form the Aliskiren granulate.

Particularly preferable is a roller compaction method whereby the step of compacting is performed using a roller compactor. In this case, the compacting step can be accomplished using any suitable means. Typically, compacting is accomplished using a roller compactor with a compaction force (for development scale machines) ranging from about 2 kN to about 6 kN i.O., preferably about 3 to 5 kN. Compaction may also be carried out by slugging the blended powders into large tablets that are then size-reduced. Preferably, the device used is a Freund Corporation; Roller Compactor Type TF Mini. Using this equipment, the screw speed is suitably adjusted to ensure proper quality of the roller compacted material. Preferably, the screw speed is more than 15 rpm, such as 20 to 30 rpm. Moreover, using this equipment, the roll speed is suitably adjusted to ensure proper quality of the roller compacted material. Preferably, the roll speed is 3 to 5 rpm. It is also preferred that no pre-compression force is applied.

In another preferred embodiment component a) is granulated by a melt extrusion granulation method. Aliskiren melt extrusion granulation is typically accomplished by using the following method: (1) blending component a) and pharmaceutically acceptable additives to form a blended material; (2) sieving the blended material, (3) melt extruding the sieved material, (4) cooling the extrudate to ambient temperature, (5) milling the melt granulation material, and (6) blending the milled melt granulation material with sieved further pharmaceutically acceptable additives to give the final Aliskiren melt granulate. Melt extrusion of Aliskiren is described, for example, in PCT/US08/077416 and in U.S. 61/099,595.

Attention is drawn to the numerous known methods of granulating, drying sieving and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press. The blending steps can be accomplished using any suitable means. Typically the component a) and pharmaceutically acceptable additives are dispatched to a suitable vessel such as a diffusion blender or diffusion mixer. The drying of step can be accomplished using any suitable means. The sieving steps can be accomplished using any suitable means, e.g. using oscillating sieving. The screening step can be accomplished using any suitable means. The compacting step can be accomplished using any suitable means. Typically compacting is accomplished using a roller compactor with a compaction force ranging of from 20 kN to 60 kN, preferably 35 kN. Compaction may also be carried out by slugging the blended powders into large tablets that are then size-reduced. The milling step can be accomplished using any suitable means. Typically the compacted material is milled through a screening mill. Preferably the milled material is blended, often with a pharmaceutically acceptable additive such as a lubricant, in a diffusion blender.

In the second step of the method, component b) is granulated with pharmaceutically acceptable additives to form a Valsartan granulate. Valsartan granulation can be accomplished by any suitable means. In a preferred embodiment of this invention, Valsartan granulation is accomplished by (1) blending component b) and pharmaceutically acceptable additives to form a blended material; (2) sieving the blended material; (3) blending the sieved material to form a final blend material; (4) compacting the final blend material to form a compacted material; (5) milling the compacted material to get a milled material; and (6) blending the milled material to form the Valsartan granulate.

The blending of step (1 and 3) can be accomplished using any suitable means. Typically the component b) and pharmaceutically acceptable additives are dispatched to a suitable vessel such as a diffusion blender or diffusion mixer. The sieving of step (2) can be accomplished using any suitable means such as those described above. The compaction of step (4) can be accomplished using any suitable means. For example, typically for component b) compacting is accomplished using a roller compactor with a compaction force ranging of from 20 kN to 60 kN, preferably 35 kN. Compaction may also be carried out by slugging the blended powders into large tablets that are then size-reduced. The milling of step (5) can be accomplished using any suitable means. Typically the compacted material is milled through a screening mill. The blending of step (6) can be accomplished using any suitable means. Preferably the milled material is blended, often with a pharmaceutically acceptable additive such as a lubricant, in a diffusion blender.

In a further step of the method, pharmaceutically acceptable additives may be added to the valsartan granulates and/or the aliskiren granulates. This is described as adding additives in the outer phase. The respective Aliskiren and Valsartan granulates are referred to as the inner phase. The additives may be distributed partly in the granulate (in the inner phase) and partly in the outer phase, which is preferably the case in the described invention. Filler, lubricant and glidant (if present), more preferably lubricant, can be distributed partly in the inner and partly in the outer phase, binder (if present) is preferably only part of the inner phase.

In one embodiment, both components a) and b) are granulated by roller compaction.

In the final step of the method, the Valsartan granulate (including additives) and the Aliskiren granulates (including additives) are compressed together to form a bilayer tablet. Compression can be accomplished using any suitable means. Typically compression is accomplished using a bilayer rotary tablet press. Typical compression force ranges of from 5 kN to 35 kN. Preferably, the layer containing component b) is pre-compressed and the layer containing component a) is added to the resulting pre-compressed layer and then both layers are compressed.

Optionally, the method comprises the step of film coating the multilayer, preferably bilayer, tablet. The details regarding the film coating material, i.e., components, amounts, etc., are as described herein. Film coating can be accomplished using any suitable means. Suitable film coatings are known and commercially available or can be made according to known methods. Typically the film coating material is a polymeric film coating material comprising materials such as polyethylene glycol, talc and colorant. Typically, a film coating material is applied in such an amount as to provide a film coating that ranges of from 1% to 6% by weight of the film-coated tablet.

The resulting formulations in accordance with the present invention show the following advantages:
Matching dissolution profile of valsartan in the presence of aliskiren to free valsartan,
The formulation of pharmaceutical oral fixed dose combinations with sufficient hardness, resistance to friability, disintegration time etc. is possible;
A robust manufacturing process is achieved;
Scale-up of formulation and process resulting in a reproducible performance is achieved; and
Sufficient stability to achieve a reasonable shelf life is achieved.

The invention likewise relates to a process for the preparation of pharmaceutical oral fixed dose combinations as described herein above. Such pharmaceutical oral fixed dose combination may be produced by working up components as defined herein above in the appropriate amounts, to form unit pharmaceutical oral fixed dose combinations.

The pharmaceutical oral fixed dose combinations of the present invention are useful for lowering the blood pressure, either systolic or diastolic or both. The conditions for which the instant invention is useful include, without limitation, hypertension (whether of the malignant, essential, reno-vascular, diabetic, isolated systolic, or other secondary type), congestive heart failure, angina (whether stable or unstable), myocardial infarction, atherosclerosis, diabetic nephropathy, diabetic cardiac myopathy, renal insufficiency, peripheral vascular disease, left ventricular hypertrophy, cognitive dysfunction (such as Alzheimer's) and stroke, headache and chronic heart failure.

The present invention likewise relates to a method of treating hypertension (whether of the malignant, essential, reno-vascular, diabetic, isolated systolic, or other secondary type), congestive heart failure, angina (whether stable or unstable), myocardial infarction, atherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure comprising administering to an animal, including human patient, in need of such treatment a therapeutically effective pharmaceutical oral fixed dose combination according to the present invention.

The present invention likewise relates to the use of a pharmaceutical oral fixed dose combination according to the present invention for the manufacture of a medicament for the treatment of hypertension (whether of the malignant, essential, reno-vascular, diabetic, isolated systolic, or other secondary type), congestive heart failure, angina (whether stable or unstable), myocardial infarction, atherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure.

The present invention likewise relates to a pharmaceutical composition for the treatment of hypertension (whether of the malignant, essential, reno-vascular, diabetic, isolated systolic, or other secondary type), congestive heart failure, angina (whether stable or unstable), myocardial infarction, atherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure, comprising a pharmaceutical oral fixed dose combination according to the present invention.

Ultimately, the exact dose of the active agent and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

Bilayer Tablet Formulations

The components of the Aliskiren layer were mixed and granulated by roller compaction, as described herein. The components of the Valsartan layer were mixed and granulated by roller compaction, as described herein. The Valsartan layer was filled into an eccentric tablet press for all bilayer variants and compressed with a compression force of <2.5 kN. The Aliskiren layer was added on top of the Valsartan layer and then the tablet core was compressed between 5-40 kN to obtain a bilayer tablet core.

### EXAMPLE 1

| | VARIANT 1 | | VARIANT 2 | |
|---|---|---|---|---|
| Aliskiren/Valsartan | | % tablet | | % tablet |
| 300/320 mg | mg per unit | weight | mg per unit | weight |
| Aliskiren layer | 600.00 | 49.18 | 600.00 | 49.18 |
| Aliskiren compacted | | | | |
| granulate | | | | |
| Aliskiren hemifumarate | 331.5 | 27.17 | 331.5 | 27.17 |
| Cellulose MK GR | 104.7 | 8.58 | 127.5 | 10.45 |
| Mannitol DC | 102 | 8.36 | 102 | 8.36 |
| Crospovidone | 18 | 1.48 | 18 | 1.48 |
| HPC EXF | 22.8 | 1.87 | — | — |
| Indigotin LAKE 12196 | 0.6 | 0.05 | 0.6 | 0.05 |
| (C) | | | | |
| Aerosil 200 | 5.7 | 0.47 | 5.7 | 0.47 |
| Mg stearate (internal) | 11.7 | 0.96 | 11.7 | 0.96 |
| Mg-Stearate (external) | 3 | 0.25 | 3 | 0.25 |
| Valsartan layer | 620.00 | 50.82 | 620.00 | 50.82 |
| Valsartan compacted | | | | |
| Granulate | | | | |
| Valsartan | 320 | 26.23 | 320.00 | 26.23 |
| Cellulose MK GR | 152 | 12.46 | 183.00 | 15.00 |
| PVP XL | 62 | 5.08 | 31.00 | 2.54 |
| L-HPC (low substituted | 62 | 5.08 | 62.00 | 5.08 |
| HPC) | | | | |
| Aerosil 200 | 6 | 0.49 | 6.00 | 0.49 |
| Mg stearate (internal) | 12 | 0.98 | 12.00 | 0.98 |
| Mg-Stearate (external) | 6 | 0.49 | 6.00 | 0.49 |
| | 1220.00 | 100.00 | 1220.00 | 100.00 |
| Hardness [N] (mean) | 300 | | 300 | |
| Friability 10St./6.5 g | 0.29 | | 0.32 | |
| 500 U. [%] | | | | |
| Disintegration time in | 1′00-1′30 | | 1′00-1′30 | |
| min/Valsartan layer | | | | |
| Disintegration time in | 23′ | | 17′ | |
| min/Aliskiren layer | | | | |

### EXAMPLE 2

| Aliskiren/Valsartan | | % tablet |
|---|---|---|
| 150/160 mg | mg per unit | weight |
| Aliskiren layer | 298.50 | 48.93 |
| Aliskiren compacted | | |
| granulate | | |
| Aliskiren hemifumarate | 165.75 | 27.17 |
| Cellulose MK GR | 63.975 | 10.49 |
| Mannitol DC | 51.00 | 8.36 |
| Indigotin Lake 12196 | 0.075 | 0.01 |
| PVP XL | 9.00 | 1.48 |
| Aerosil 200 | 2.85 | 0.47 |
| Mg stearate (internal) | 5.85 | 0.96 |
| Mg-Stearate (external) | 1.50 | 0.25 |
| Valsartan layer | 307.00 | 50.33 |
| Valsartan compacted | | |
| Granulate | | |
| Valsartan | 160.00 | 26.23 |
| Cellulose MK GR | 91.50 | 15.00 |
| PVP XL | 15.50 | 2.54 |
| L-HPC | 31.00 | 5.08 |
| Aerosil 200 | 3.00 | 0.49 |
| Mg stearate (internal) | 6.00 | 0.98 |
| Mg-Stearate (external) | 3.00 | 0.21 |
| | 610.00 | 100.00 |
| Hardness [N] (mean) | 270 | |
| Friability 10St./6.5 g | 0.3 | |
| 500 U. [%] | | |
| Disintegration Time | 16.5 | |
| (min) | | |

### EXAMPLE 3

| Aliskiren/Valsartan | | % tablet |
|---|---|---|
| 300/320 mg | mg per unit | weight |
| Aliskiren layer | 597.00 | 48.93 |
| Aliskiren compacted | | |
| granulate | | |
| Aliskiren hemifumarate | 331.50 | 27.17 |
| Cellulose MK GR | 127.95 | 10.49 |
| Mannitol DC | 102.00 | 8.36 |
| Indigotin Lake 12196 | 0.15 | 0.01 |
| PVP XL | 18.00 | 1.48 |
| Aerosil 200 | 5.70 | 0.47 |
| Mg stearate (internal) | 11.70 | 0.96 |
| Mg-Stearate (external) | 3.00 | 0.25 |
| Valsartan layer | 614.00 | 50.33 |
| Valsartan compacted | | |
| Granulate | | |
| Valsartan | 320..00 | 26.23 |
| Cellulose MK GR | 183.00 | 15.00 |
| PVP XL | 31.00 | 2.54 |
| L-HPC | 62.00 | 5.08 |
| Aerosil 200 | 6.00 | 0.49 |
| Mg stearate (internal) | 12.00 | 0.98 |
| Mg-Stearate (external) | 6.00 | 0.21 |
| | 1220.00 | 100.00 |
| Hardness [N] (mean) | 300 | |
| Friability 10St./6.5 g | 0.4 | |
| 500 U. [%] | | |
| Disintegration Time | 17.1 | |
| (min) | | |

### EXAMPLE 4

| | VARIANT | | VARIANT | | VARIANT | |
|---|---|---|---|---|---|---|
| | 1 | | 2 | | 3 | |
| Aliskiren/Valsartan | mg per | % tablet | mg per | % tablet | mg per | % tablet |
| 300/320 mg | unit | weight | unit | weight | unit | weight |
| Aliskiren layer | 600.00 | 49.18 | 520 | 45.61 | 600 | 49.18 |
| Aliskiren compacted | | | | | | |
| granulate | | | | | | |
| Aliskiren hemifumarate | 331.5 | 27.17 | 331.5 | 29.08 | 331.5 | 27.17 |
| Cellulose MK GR | 172.5 | 14.14 | 105.3 | 9.24 | 69.3 | 5.68 |
| Mannitol DC | 48 | 3.93 | 41.6 | 3.65 | 132 | 10.82 |
| Crospovidone | 12 | 0.98 | 10.4 | 0.91 | 16.2 | 1.33 |
| HPC EXF | 18 | 1.48 | 15.6 | 1.37 | 30 | 2.46 |
| Indigotin LAKE 12196 (C) | — | — | — | — | 0.6 | 0.05 |
| Aerosil 200 | 3 | 0.25 | 2.6 | 0.23 | 5.7 | 0.47 |
| Mg stearate (internal) | 12 | 0.98 | 10.4 | 0.91 | 11.7 | 0.96 |
| Mg-Stearate (external) | 3 | 0.25 | 2.6 | 0.23 | 3 | 0.25 |
| Valsartan layer | 620.00 | 50.82 | 620.00 | 54.39 | 620.00 | 50.82 |
| Valsartan compacted | | | | | | |
| Granulate | | | | | | |
| Valsartan | 320 | 26.23 | 320 | 28.07 | 320 | 26.23 |
| Cellulose MK GR | 152 | 12.46 | 152 | 13.33 | 152 | 12.46 |
| PVP XL | 62 | 5.08 | 62 | 5.44 | 62 | 5.08 |
| L-HPC (low substituted | 62 | 5.08 | 62 | 5.44 | 62 | 5.08 |
| HPC) | | | | | | |
| Aerosi1200 | 6 | 0.49 | 6 | 0.53 | 6 | 0.49 |
| Mg stearate (internal) | 12 | 0.98 | 12 | 1.05 | 12 | 0.98 |
| Mg-Stearate (external) | 6 | 0.49 | 6 | 0.53 | 6 | 0.49 |
| | 1220.00 | 100.00 | 1140.00 | 100.00 | 1220.00 | 100.00 |
| Hardness [N] (mean) | 288 | | 275 | | 278 | |
| Friability 10 St./6.5 g | 0.17 | | 0.37 | | 0.39 | |
| 500 U. [%] | | | | | | |
| Disintegration time in | 1′00-1′30 | | 1′00-1′30 | | 1′00-1′30 | |
| min/Valsartan layer | | | | | | |
| Disintegration time in | 23′ | | 22′ | | 21′30-25′15 | |
| min/Aliskiren layer | | | | | | |

### EXAMPLE 5

| | VARIANT 1 | | VARIANT 2 | |
|---|---|---|---|---|
| Aliskiren/Valsartan | | % tablet | | % tablet |
| 300/320 mg | mg per unit | weight | mg per unit | weight |
| Aliskiren layer | 600.00 | 49.18 | 520.00 | 45.61 |
| Aliskiren compacted | | | | |
| granulate | | | | |
| Aliskiren hemifumarate | 331.5 | 27.17 | 331.5 | 29.08 |
| Cellulose MK GR | 104.7 | 8.58 | 47.84 | 4.20 |
| Mannitol DC | 102 | 8.36 | — | — |
| Lactose, Anhydrous DT | — | — | 83.2 | 7.30 |
| Crospovidone | 18 | 1.48 | 10.4 | 0.91 |
| HPC EXF | 22.8 | 1.87 | 31.2 | 2.74 |
| Indigotin LAKE 12196 | 0.6 | 0.05 | 0.6 | 0.05 |
| (C) | | | | |
| Aerosil 200 | 5.7 | 0.47 | 4.94 | 0.43 |
| Mg stearate (internal) | 11.7 | 0.96 | 7.8 | 0.68 |
| Mg-Stearate (external) | 3 | 0.25 | 2.6 | 0.23 |
| Valsartan layer | 620.00 | 50.82 | 620.00 | 54.39 |
| Valsartan compacted | | | | |
| Granulate | | | | |
| Valsartan | 320 | 26.23 | 320 | 28.07 |
| Cellulose MK GR | 152 | 12.46 | 152 | 13.33 |
| PVP XL | 62 | 5.08 | 62 | 5.44 |
| L-HPC (low substituted | 62 | 5.08 | 62 | 5.44 |
| HPC) | | | | |
| Aerosil 200 | 6 | 0.49 | 6 | 0.53 |
| Mg stearate (internal) | 12 | 0.98 | 12 | 1.05 |
| Mg-Stearate (external) | 6 | 0.49 | 6 | 0.53 |
| | 1220.00 | 100.00 | 1140.00 | 100.00 |
| Hardness [N] (mean) | 300 | | 221 | |
| Friability 10St./6.5 g | 0.29 | | 0.20 | |
| 500 U. [%] | | | | |
| Disintegration time in | 1′00-1′30 | | 1′00-1′30 | |
| min/Valsartan layer | | | | |
| Disintegration time in | 22′45″ | | 24′-26′ | |
| min/Aliskiren layer | | | | |

Dissolution Testing

### EXAMPLE 6

The dissolution property of the formulations in accordance with the present invention were confirmed as follows.

Paddle method at pH 4.5: The assembly consists of the following: a covered vessel made of glass or other inert, transparent material; a motor, and a paddle formed from a blade and shaft as the stirring element. The vessel is partially immersed in a suitable water bath of any convenient size or placed in a heating jacket. The water bath or heating jacket permits holding the temperature inside the vessels at 37±0.5° during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test has the following dimensions and capacities: the height is 160 mm to 210 mm and its inside diameter is 98 mm to 106 mm. Its sides are flanged at the top. A fitted cover may be used to retard evaporation.

The shaft is positioned so that its axis is not more than 2 mm at any point from the vertical axis of the vessel and rotates smoothly without significant wobble. The vertical center line of the blade passes through the axis of the shaft so that the bottom of the blade is flush with the bottom of the shaft. The design of the paddle is as shown in USP <711>, FIG. 2. The distance of 25±2 mm between the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable inert coating. The dosage unit is allowed to sink to the bottom of the vessel before rotation of the blade is started. A small, loose piece of nonreactive material such as not more than a few turns of wire helix may be attached to dosage units that would otherwise float. Other validated sinker devices may be used.

One liter of the Dissolution Medium* is placed in the vessel of the apparatus, the apparatus is assembled, the Dissolution Medium is equilibrated to 37±0.5°, and the thermometer is removed. One dosage form (e.g. tablet or capsule) is placed on the apparatus, taking care to exclude air bubbles from the surface of the dosage-form unit, and immediately the apparatus is operated at a rate of 75±3 rpm or 100±3rpm depending on the pH. Within the time interval specified (e.g. 10, 20, 30, 45, 60, 90 and 120 min.), or at each of the times stated, a specimen (≧1 ml) is withdrawn from a zone midway between the surface of the Dissolution Medium and the top of the rotating blade, not less than 1 cm from the vessel wall. [NOTE—the aliquots withdrawn for analysis are replaced with equal volumes of fresh Dissolution Medium at 37° or, where it can be shown that replacement of the medium is not necessary, the volume change is corrected in the calculation. The vessel is kept covered for the duration of the test, and the temperature of the mixture under test at suitable times is verified.] The specimen is filtered through a suitable filter, e.g. a 0.45 μm PVDF filter (Millipore) and the first mls (2 to 3 ml) of the filtrate are discarded. The analysis is performed by HPLC or UV detection. The test is repeated at least six times with additional dosage form units.

* Dissolution medium for pH 4.5: One liter of a buffered aqueous solution, adjusted to pH 4.5±0.05 (0.1 M Phosphate buffer solution obtained by dissolving 13.61 g of potassium hydrogen phosphate in 750 ml of deionized water and diluted to 1 L with deionized water)

The examples of pharmaceutical oral fixed dose combinations of the present invention prepared according to the present invention all had the required dissolution characteristics as set forth in the claims of the present invention. The results are shown in the table below.

| | Dissolution | Dissolution | Dissolution | Dissolution |
|---|---|---|---|---|
| | profile of | profile of | profile of | profile of |
| | Aliskiren | Aliskiren | Valsartan | Valsartan |
| | at pH 4.5 | at pH 4.5 | at pH 4.5 | at pH 4.5 |
| | after 10 min | after 20 min | after 30 min | after 60 min |
| Example 1 | 32.1 | 59.1 | 65.4 | 77.9 |
| Variant 1 | | | | |
| Example 1 | 40.11 | 76.15 | 58.89 | 75.30 |
| Variant 2 | | | | |
| Example 2 | 44.96 | 98.33 | 61.15 | 87.29 |
| Example 3 | 40.11 | 76.15 | 58.89 | 75.30 |
| Example 4 | 36.1 | 63.2 | 60.96 | 74.56 |
| Variant 1 | | | | |
| Example 4 | 41.5 | 69.0 | 59.89 | 73.64 |
| Variant 2 | | | | |
| Example 4 | 32.6 | 57.9 | 58.05 | 72.18 |
| Variant 3 | | | | |
| Example 5 | 32.1 | 59.1 | 65.4 | 77.9 |
| Variant 1 | | | | |
| Example 5 | 30.7 | 59.5 | 64.8 | 77.2 |
| Variant 2 | | | | |

Bioequivalence of Free Combination and Fixed-Dose Combination

### EXAMPLE 7

An open-label, randomized, two-treatment, crossover, single-dose study to determine the bioequivalence of fixed combination of aliskiren/valsartan 300/320 mg tablet and the free combination of aliskiren 300 mg and valsartan 320 mg was performed in 78 healthy subjects. The fixed combination tablet of 300/320 mg aliskiren/valsartan was bioequivalent to the free combination of 300 mg aliskiren and 2×160 mg valsartan capsules. The 90% confidence intervals of geometric mean ratios for AUC/Cmax of both aliskiren and valsartan were contained within the bioequivalence limits of 0.80-1.25, which indicates that the test formulation is bioequivalent to the reference formulation. The rate and extent of absorption of aliskiren and valsartan from the fixed combination of 300/320 mg aliskiren/valsartan tablet was similar to that from the free combination of a 300 mg aliskiren tablet and two 160 mg valsartan capsules. Both the free and fixed combinations were safe and well-tolerated.

Pharmacokinetic measurements were performed on blood collected from each subject. A combined LC/MS/MS method was used to detect aliskiren and valsartan in the same plasma sample. The lower limit of quantitation was 0.5 ng/ml for aliskiren and 5.0 ng/ml for valsartan. The PK parameters were determined in plasma, using non-compartmental methods.

Log-transformed AUC₀₋ₜₗₐₛₜ, AUC_{0-inf} and Cₘₐₓ measurements of aliskiren and valsartan were analyzed separately using a linear mixed effects model. The following pharmacokinetic methods were determined for aliskiren and valsartan.

AUC₀₋ₜₗₐₛₜ: Area under the concentration-time curve from time zero to time tlast, where tlast is the last time point with measurable concentration (ng hr/ml).

AUC_{0-inf}: Area under the plasma concentration-time curve from time zero to infinity (ng hr/ml).

Cₘₐₓ: Maximum (peak) plasma concentration (ng/ml).

Tₘₐₓ: Time to reach peak or maximum concentration (hr).

T_{1/2}: Elimination half-life associated with the terminal slope (E) of a semilogarithmic concentration-time curve (hr).

Statistical Analysis of PK Parameters

The data in the following table shows that AUC and Cₘₐₓ were contained within the equivalence limits of 0.8-1.25 for both aliskiren and valsartan. This demonstrates that the fixed combination of 300/320 mg aliskiren/valsartan tablet was bioequivalent to the free combination of a 300 mg aliskiren tablet and two 160 mg valsartan capsules.

| | Adjusted | | Ratio of | |
|---|---|---|---|---|
| | geometric means | | geometric means | |
| PK | | Reference | | 90% Confidence |
| Parameter | Test (N) | (N) | Estimate | Interval |
| Aliskiren | | | | |
| Cₘₐₓ (ng/ml) | 159.44 (80) | 164.39 (83) | 0.97 | 0.85-1.10 |
| AUC₀₋ₜₗₐₛₜ | 792.13 (79) | 797.17 (83) | 0.99 | 0.91-1.08 |
| (ng hr/ml) | | | | |
| AUC_{0-inf} | 859.32 (77) | 860.73 (83) | 1.00 | 0.92-1.09 |
| (ng hr/ml) | | | | |
| Valsartan | | | | |
| Cₘₐₓ (ng/ml) | 3833.28 (80) | 3532.28 (83) | 1.09 | 0.98-1.20 |
| AUC₀₋ₜₗₐₛₜ | 31729.8 (79) | 29204.2 (83) | 1.09 | 1.01-1.17 |
| (ng hr/ml) | | | | |
| AUC_{0-inf} | 32657.2 (74) | 29529.5 (80) | 1.11 | 1.02-1.19 |
| (ng hr/ml) | | | | |

The intra-subject coefficients of variation (CV) for AUC₀₋ₜₗₐₛₜ, AUC_{0-inf} and Cₘₐₓ of aliskiren were 33.98%, 33.19% and 51.90%, respectively and the intra-subject CV for AUC₀₋ₜₗₐₛₜ, AUC_{0-inf} and Cₘₐₓ of valsartan were 28.56%, 28.33% and 40.37%, respectively.

Aliskiren PK: Free Combination and Fixed Dose Combination with Valsartan

The mean plasma concentration-time profiles of aliskiren were similar following single oral doses of 300/320 mg aliskiren/valsartan fixed combination tablet compared to those obtained following administration of the free combination of an aliskiren 300 mg tablet and two 160 mg valsartan capsules. The geometric mean ratios (90% CI) for AUC₀₋ₜₗₐₛₜ and Cₘₐₓ were 0.99 (0.91-1.08) and 0.97 (0.85-1.10), respectively. The inter-subject variability (% CV) associated with AUC and Cₘₐₓ in both treatments was similar. Mean half-life and median Tₘₐₓ were also similar between the treatments.

| Treat- | | AUC_{0-inf} | AUC₀₋ₜₗₐₛₜ | Cₘₐₓ | Tₘₐₓ | T_{1/2} |
|---|---|---|---|---|---|---|
| ment | | (ng hr/ml) | (ng hr/ml) | (ng/ml) | (hr) | (hr) |
| Test | N | 77 | 79 | 80 | 80 | 77 |
| | Mean | 955.38 | 879.64 | 183.79 | 1.27 | 33.81 |
| | SD | 515.82 | 478.10 | 108.01 | 0.86 | 9.53 |
| | Min | 382.24 | 353.91 | 48.70 | 0.48 | 17.12 |
| | Median | 840.70 | 772.42 | 154.50 | 1.00 | 32.32 |
| | Max | 3839.88 | 3600.60 | 595.00 | 4.00 | 86.43 |
| | % CV | 54 | 54 | 59 | 68 | 28 |
| Refer- | N | 83 | 83 | 83 | 83 | 83 |
| ence | | | | | | |
| | Mean | 1005.32 | 933.19 | 202.49 | 1.16 | 33.63 |
| | SD | 673.71 | 626.74 | 144.94 | 0.83 | 8.20 |
| | Min | 323.16 | 296.67 | 46.40 | 0.47 | 13.95 |
| | Median | 803.29 | 763.81 | 163.00 | 1.00 | 32.58 |
| | Max | 4650.05 | 4248.63 | 858.00 | 4.00 | 55.40 |
| | % CV | 67 | 67 | 72 | 72 | 24 |

Valsartan PK: Free Combination and Fixed Dose Combination with Aliskiren

The mean plasma concentration-time profiles of aliskiren were similar following single oral doses of 300/320 mg aliskiren/valsartan fixed combination tablet compared to those obtained following administration of the free combination of an aliskiren 300 mg tablet and two 160 mg valsartan capsules. The geometric mean ratios (90% CI) for AUC₀₋ₜₗₐₛₜ and Cₘₐₓ were 1.09 (1.01-1.17) and 1.09 (0.98-1.20), respectively. The inter-subject variability (% CV) associated with AUC and Cₘₐₓ in both treatments was similar. Mean half-life and median Tₘₐₓ were also similar between the treatments.

| Treat- | | AUC_{0-inf} | AUC₀₋ₜₗₐₛₜ | Cₘₐₓ | Tₘₐₓ | T_{1/2} |
|---|---|---|---|---|---|---|
| ment | | (ng hr/ml) | (ng hr/ml) | (ng/ml) | (hr) | (hr) |
| Test | N | 74 | 79 | 80 | 80 | 74 |
| | Mean | 35468.81 | 34421.91 | 4391.13 | 3.44 | 12.41 |
| | SD | 13652.96 | 13676.31 | 2072.49 | 1.43 | 4.94 |
| | Min | 6414.57 | 5911.43 | 345.00 | 1.00 | 5.66 |
| | Median | 33919.05 | 32916.35 | 4275.00 | 3.01 | 11.40 |
| | Max | 74819.16 | 744.8.20 | 9140.00 | 12.00 | 25.68 |
| | % CV | 39 | 40 | 47 | 42 | 40 |
| Refer- | N | 80 | 83 | 83 | 83 | 80 |
| ence | | | | | | |
| | Mean | 31845.23 | 31509.84 | 3995.08 | 3.48 | 11.80 |
| | SD | 12262.22 | 12315.41 | 1955.46 | 1.11 | 5.21 |
| | Min | 8766.14 | 8598.03 | 797.00 | 1.50 | 5.60 |
| | Median | 29509.24 | 29287.13 | 3670.00 | 4.00 | 9.95 |
| | Max | 85004.81 | 84872.17 | 11800.00 | 6.02 | 34.64 |
| | % CV | 39 | 39 | 49 | 32 | 44 |

1-22. (canceled)

23. A pharmaceutical oral fixed dose solid combination bilayer tablet comprising
a) a first layer comprising a therapeutically effective amount of aliskiren, or a pharmaceutically acceptable salt thereof and
b) a second layer comprising a therapeutically effective amount of valsartan, or a pharmaceutically acceptable salt thereof, further comprising
(i) crospovidone, and
(ii) a polysaccharide chosen from a starch, a starch derivative and a cellulose derivative.

24. The tablet of claim 23, wherein the starch or starch derivative is a polycarboxyalkyl ether starch, a polycarboxymethyl ether starch, sodium starch glycolate, alginate or a pregelatinized starch.

25. The tablet of claim 23, wherein the polysaccharide comprises glucose or uronic residues.

26. The tablet of claim 23, wherein the cellulose derivative is a polycarboxyalkyl ether cellulose, a polycarboxymethyl ether cellulose, a low-substituted poly(hydroxylalkyl)ether of cellulose, a poly(hydroxypropyl)ether of cellulose, croscarmellose sodium or a low-substituted hydroxypropyl cellulose.

27. The tablet of claim 23, wherein the weight ratio of valsartan or the pharmaceutically acceptable salt thereof to component (ii) is from about 15:1 to about 2:1.

28. The tablet of claim 27, wherein the weight ratio of valsartan or the pharmaceutically acceptable salt thereof to component (ii) is from about 8:1 to about 2:1.

29. The tablet of claim 28, wherein the weight ratio of valsartan or the pharmaceutically acceptable salt thereof to component (ii) is from about 6:1 to about 3:1.

30. The tablet of claim 23, wherein the weight ratio of (i) to (ii) is from about 1:1 to about 1:8.

31. The tablet of claim 30, wherein the weight ratio of (i) to (ii) is from about 1:1 to about 1:5.

32. The tablet of claim 31, wherein the weight ratio of (i) to (ii) is from about 1:1 to about 1:3.

33. The tablet of claim 23, wherein the second layer comprises (ii) in an amount of from about 2 to about 20%, prior to an optional film coating.

34. The tablet of claim 33, wherein the second layer comprises (ii) in an amount of from about 4 to 10%, prior to an optional film coating.

35. The tablet of claim 34, wherein the second layer comprises (ii) in an amount of from about 4 to 6%, prior to an optional film coating.

36. The tablet of claim 23, wherein the first layer comprises crospovidone.

37. The tablet of claim 36, wherein the weight ratio of aliskiren, or the pharmaceutically acceptable salt thereof to crospovidone is of from about 40:1 to about 10:1.

38. The tablet of claim 37, wherein the weight ratio of aliskiren, or the pharmaceutically acceptable salt thereof to crospovidone is from about 30:1 to about 10:1.

39. The tablet of claim 38, wherein the weight ratio of aliskiren, or the pharmaceutically acceptable salt thereof to crospovidone is from about 30:1 to about 15:1.

40. The tablet of claim 36, wherein the weight ratio of (ii) to crospovidone is from about 15:1 to about 3:1.

41. The tablet of claim 40, wherein the weight ratio of (ii) to crospovidone is from about 10:1 to 3:1.

42. The tablet of claim 41, wherein the weight ratio of (ii) to crospovidone is from about 7:1 to about 3:1.

43. The tablet of claim 23, wherein aliskiren, or the pharmaceutically acceptable salt thereof is present in an amount of from about 75 to about 300 mg free base.

44. The tablet of claim 23, wherein valsartan, or the pharmaceutically acceptable salt thereof is present in an amount of from about 80 to 320 mg.

45. The tablet of claim 23, wherein the first layer is obtained by roller compaction.

46. The tablet of claim 23, wherein the second layer is obtained by roller compaction.

47. A method of using a pharmaceutical oral fixed dose solid combination bilayer tablet comprising
a) a first layer comprising a therapeutically effective amount of aliskiren, or a pharmaceutically acceptable salt thereof and
b) a second layer comprising a therapeutically effective amount of valsartan, or a pharmaceutically acceptable salt thereof, further comprising
(i) crospovidone, and
(ii) a polysaccharide chosen from a starch, a starch derivative and a cellulose derivative to treat hypertension.

48. A method of producing the tablet of claim 23 comprising
(a) granulating aliskiren or the pharmaceutically acceptable salt thereof and pharmaceutically acceptable additives to form an aliskiren granulate;
(b) granulating valsartan or the pharmaceutically acceptable salt thereof, crospovidone, a polysaccharide chosen from a starch, a starch derivative and a cellulose derivative, and pharmaceutically acceptable additives to form a valsartan granulate;
(c) optionally drying granulates (a) and/or (b);
(d) sieving;
(e) optionally mixing granulates (a) and/or (b) with outer phase excipients; and
(f) compressing granulates (a) and (b) together to form a bilayer tablet.
